(19) United States
(12) Patent Application Publication
(10) Pub. No.: US 2026/0097163 A1
(43) Pub. Date: Apr. 9, 2026

(54) PRESSURE BALANCING ACROSS MICROFLUIDIC DEVICES

(71) Applicant: Astrin Biosciences, Inc., St. Paul, MN (US)

(72) Inventors: Mahdi Ahmadi, Minneapolis, MN (US); Kaylee Judith Kamalanathan, Woodbury, MN (US); Jiarong Hong, Eden Prairie, MN (US); Jayant Parthasarathy, Excelsior, MN (US)

(73) Assignee: Astrin Biosciences, Inc., St. Paul, MN (US)

(21) Appl. No.: 19/113,966

(22) PCT Filed: Sep. 22, 2023
(86) PCT No.: PCT/US2023/074945

§ 371 Date: Mar. 21, 2025

Related U.S. Application Data
(60) Provisional application No. 63/408,962, filed on Sep. 22, 2022.
(60) Provisional application No. 63/408,963, filed on Sep. 22, 2022.
(60) Provisional application No. 63/408,972, filed on Sep. 22, 2022.

(51) Int. Cl. A61M 1/38; A61B 10/02; A61M 1/36

(52) U.S. Cl. CPC A61M 1/38; A61B 10/02; A61M 1/36225; A61M 2205/3337

(57) ABSTRACT

The present aspects relate to an aphaeretic biopsy system. The aphaeretic biopsy system can implement a dialysis process to obtain a bloodstream by a draw line, separate released tumor cells (RTCs) from the bloodstream, capture the RTCs in a reservoir for further analysis, and return the remaining portion of the bloodstream to the patient via a return line. The system can include a removable cartridge that comprises a stack of microfluidic devices configured to implement inertial separation techniques to separate the RTCs from the bloodstream. The stack of microfluidic devices can implement pressure balancing techniques to balance a pressure across multiple devices. The pressure balancing can allow for an even distribution of blood across multiple microfluidic devices and increased performance in separating RTCs from the bloodstream.

### PRIORITY

This application claims the benefit of U.S. Ser. No. 63/408,962, filed on Sep. 22, 2022, U.S. Ser. No. 63/408,972, filed on Sep. 22, 2022, and U.S. Ser. No. 63/408,963, filed on Sep. 22, 2022, the contents of which are incorporated by reference herein in their entirety.

### TECHNICAL FIELD

The methods and compositions described here relate to the field of an aphaeretic biopsy system. Some aspects relate to an aphaeretic biopsy device using one or more microfluidic devices where the entire blood volume is processed inside a disposable cartridge including one or more microfluidic devices.

### BACKGROUND

Inertial microfluidic techniques can generally be used to separate particles based on a size of the particles. For example, an inertial microfluidic device can obtain a number of microparticles with varying flow rates and separate the microparticles by size.

Devices incorporating inertial microfluidic techniques can operate in a laminar region and can rely on a balance of an inertial lift force and a wall force to focus particles to distinct streamlines. The location of the streamlines can vary based on any of a channel geometry and particle characteristics. For instance, in straight cylindrical channels, particles can focus to an inner circle a specific distance from the channel wall. In a square channel, the particles can focus to any of four locations equidistant to one another. By changing a cross-sectional area to be rectangular, the location can often collapse into two locations. Adding curvature can further increase an ability to optimize the focusing of various particle types to different streamlines.

### SUMMARY

The compositions, devices, and methods described herein relate to aphaeretic biopsy systems. Aphaeretic biopsy systems can implement a dialysis system to obtain a stream of blood cells, separate released tumor cells (RTCs) from the blood, and return a remaining portion of the blood to a patient. RTCs can be circulating tumor cells (CTCs), disseminated tumor cells (DTCs), or any other type of RTCs. CTCs can be tumor cells that have been shed into the vasculature or lymphatic systems. DTCs are tumor cells that have been shed from a tumor, which have subsequently landed at a secondary site in the same subject. DTCs can form a tumor at the second site in the subject. The RTCs can be captured in a reservoir for further analysis. An aphaeretic biopsy system can include a removable cartridge comprising one or more microfluidic devices configured to separate the RTCs from the blood using inertial separation.

A removable cartridge can include a stack of multiple microfluidic devices, with each device obtaining a portion of the bloodstream via a channel. A pressure in each channel can be balanced to ensure similar distribution of blood across each device using any of an active or a passive pressure balancing system. Active pressure balancing systems can include using any of a valve, pump, and/or a chamber to balance pressure across channels. Passive pressure balancing systems can include modifying a length and/or a width of each channel to balance the pressure of each channel.

In one aspect, a particle separation system comprising multiple microfluidic devices with a balanced pressure across the multiple microfluidic devices is provided. The system can include an inlet obtaining a stream of blood cells.

A system can also include a plurality of microfluidic devices each configured to obtain a portion of the stream of blood cells from the inlet. Each of the plurality of microfluidic devices can include at least one separation channel to separate particles in the stream of blood cells, at least a first outlet for capturing released tumor cells included in the stream of blood cells, and a second outlet for returning a remaining portion of the blood cells via a return line.

A system can also include a series of channels connecting the inlet to each of the plurality of microfluidic devices. Each of the channels can include a length and a width that is variable to balance a pressure of each of the channels as the stream of blood cells are directed through each channel.

In some instances, each of the series of channels include at least a first channel connecting the inlet to a first microfluidic device, the first channel comprising a first length and width. Each of the series of channels can also include a second channel connecting the inlet to a second microfluidic device, the second channel comprising a second length and width greater than that of the first channel.

In some instances, the length and width of each of the series of channels is based on a major pressure loss due to friction loss as each portion of the stream of blood cells travel through each channel. In some instances, determining the major pressure loss is based on a dynamic viscosity, the length, a volumetric flow rate, and a cross sectional area of each of the series of channels.

In some instances, the length and width of each of the series of channels is based on a fluid density, a hydraulic diameter, a mean flow velocity, and a flow coefficient of each of the series of channels.

In some instances, a system can include any of a valve, a chamber, and/or a pump connected to each channel, wherein the valve, chamber, and/or pump are configured to modify and control the pressure of each channel.

In some instances, a system can include a closed-loop control system configured to adjust the pressure of the pump based on detected pressure changes in any of the channels.

In some instances, a system can include a reservoir pump connected to a reservoir configured to capture the released tumor cells output from the first output, wherein the reservoir is configured to be pressurized.

In another example aspect, a device is provided. A device can include an inlet obtaining a stream of blood cells. A device can also include a plurality of microfluidic devices each configured to obtain a portion of the stream of blood cells from the inlet. Each of the plurality of microfluidic devices can include at least one separation channel to separate particles in the stream of blood cells, at least a first outlet for capturing released tumor cells included in the stream of blood cells, and a second outlet for returning a remaining portion of the blood cells via a return line.

A device can also include a series of channels connecting the inlet to each of the plurality of microfluidic devices. Each of the channels can include a length and a width that is variable to balance a pressure of each of the channels as the stream of blood cells are directed through each channel. A device can also include any of a valve, a chamber, and/or a pump connected to each channel. The valve, chamber, and/or pump can be configured to modify and control the pressure of each channel.

In some instances, A device can include a closed-loop control system configured to adjust the pressure of the pump based on detected pressure changes in any of the channels.

In some instances, A device can include a pump connected to a reservoir configured to capture the released tumor cells output from the first output, wherein the reservoir is configured to be pressurized.

In some instances, the length and width of each of the series of channels is based on a major pressure loss due to friction loss as each portion of the stream of blood cells travel through each channel.

In some instances, the length and width of each of the series of channels is based on a major pressure loss due to friction loss as each portion of the stream of blood cells travel through each channel.

In some instances, the length and width of each of the series of channels is based on a fluid density, a hydraulic diameter, a mean flow velocity, and a flow coefficient of each of the series of channels.

In another example aspect, methods for separating released tumor cells from a stream of blood cells via multiple microfluidic devices is provided. The methods can include obtaining, at an inlet, a stream of blood cells. The methods can also include pressurizing a series of channels using any of a valve, chamber, and/or a pump.

Methods can also include directing portions of each of the stream of blood cells through each of the series of channels to corresponding microfluidic devices. At least one separation channel for each microfluidic device can be configured to separate particles in each portion of the stream of blood cells. The methods can also include obtaining released tumor cells at a first outlet of each of the microfluidic devices. The methods can also include obtaining remaining portions of the stream of blood cells at a second outlet of each of the microfluidic devices.

In some instances, methods can include pressurizing a reservoir using a reservoir pump and capturing the released tumor cells from the first outlet at the reservoir.

In some instances, methods can include determining a pressure in each of a series of channels connecting an inlet to a plurality of microfluidic devices and modifying a length and a width for each of the series of channels based on the pressure in each of a series of channels.

In some instances, the length and width of each of the series of channels is based on a major pressure loss due to friction loss as each portion of the stream of blood cells travel through each channel.

In some instances, determining the major pressure loss is based on a dynamic viscosity, the length, a volumetric flow rate, and a cross sectional area of each of the series of channels. In some instances, the length and width of each of the series of channels is based on a fluid density, a hydraulic diameter, a mean flow velocity, and a flow coefficient of each of the series of channels.

### BRIEF DESCRIPTION OF THE DRAWINGS

Aspects are illustrated by way of example and not limitation in the figures of the accompanying drawings, in which like references indicate similar elements and in which:

FIG. 1 illustrates an example aphaeretic biopsy system according to an aspect.

FIG. 2 is an exploded view of an example cartridge according to an aspect.

FIG. 3 illustrates an example particle separation device with a sawtooth design according to an aspect.

FIG. 4 illustrates an example particle separation device with a double stage sawtooth design with a closed end outlet disposed between stages according to an aspect.

FIG. 5 illustrates an example active pressure balancing system according to an aspect.

FIG. 6 illustrates a first example passive pressure balancing system according to an aspect.

FIG. 7 illustrates an example passive pressure balancing system with varied lengths and widths of each channel according to an aspect.

FIG. 8 illustrates an example series of microfluidic devices with an active pressure balancing system according to an aspect.

FIG. 9 is a flow process of an example method for separating released tumor cells from a blood stream through a pressure balanced aphaeretic system according to an aspect.

FIG. 10 illustrates an example cartridge according to an aspect.

FIG. 11 illustrates inserting a cartridge in an outer housing as part of an aphaeretic biopsy system according to an aspect.

FIG. 12 illustrates a top view of the aphaeretic biopsy system according to an aspect.

FIG. 13 illustrates a side view of an example aphaeretic biopsy system according to an aspect.

FIG. 14 illustrates a rear view of an example aphaeretic biopsy system according to an aspect.

FIG. 15 illustrates an example method for separating released tumor cells from an aphaeretic biopsy system according to an aspect.

FIG. 16 illustrates an example method for configuring an aphaeretic biopsy system according to an aspect.

FIG. 17 is a block diagram of a special-purpose computer system 1700 according to an aspect.

### DETAILED DESCRIPTION

In many instances, cells released from a tumor in the body can travel through the bloodstream along with other blood cells. These cancer cells (otherwise known as “released tumor cells” or “circulating tumor cells”) can be released from various parts of the body from any of one or more tumors in the body.

It can be desirable to capture and analyze the released tumor cells to derive insights into the tumor cells. For example, released tumor cells can be analyzed to determine a location of the tumor in the body as well as a type of cancer. Multiple different tumors can be identified separately by analyzing released tumor cells originating from different tumors.

In many cases, a biopsy can be performed to isolate tumor cells for analysis. A biopsy can include extracting tissue from a tumor, which can be difficult and painful to a patient. Furthermore, many types of cancers and various locations of tumors can be extremely painful or nearly impossible to effectively extract as part of a biopsy.

The present aspects relate to aphaeretic biopsy systems utilizing one or more microfluidic device to separate released tumor cells (RTCs) from a bloodstream. The aphaeretic biopsy system implements a dialysis process to obtain a stream of blood from the patient (e.g., an animal, a mammal, or a human), direct the blood into a removable cartridge, and direct the bloodstream through one or more microfluidic devices. The microfluidic devices, in turn, can implement inertial separation techniques to separate released tumor cells from the bloodstream. Separation and/or enrichment of RTCs can involve inertial microfluidic technology which applies the effects of the secondary flow of the macroscopic fluid to the microscopic flow channel and thus can separate RTCs from other cell types based on their morphological differences.

The captured RTCs can be held in a reservoir and further analyzed to derive insights into the cancer cells, such as a location of one or more cancer cell clusters, a stage of the cancer, and/or a type of cancer. An aphaeretic biopsy system can include a removable cartridge and an outer housing configured to receive the cartridge.

While the methods and devices described herein can be used to draw a sample directly from a patient and return part of the sample directly to the patient, the methods and devices can be used ex vivo also. For example, the method is completed completely ex vivo using containers having previously collected samples and having the second outlet connected to a container instead of a patient.

The present methods and devices provide several advantages. For example, rather than performing an invasive biopsy to capture tissue containing cancer cells, an aphaeretic biopsy system as described herein utilize dialysis to obtain a stream of blood cells from a patient, separate RTCs from the blood cells, and return the remaining blood cells back to the patient. Such a process can be less intrusive to capture RTCs for further analysis as compared to a surgical biopsy.

Furthermore, performing a biopsy can only obtain cancer cells of one or more known cancer clusters, while missing other cancer clusters. In contrast, aphaeretic biopsy systems as described herein can obtain RTCs from any number of cancer clusters, providing greater insights into the cancer clusters in the body. Particularly with types of cancers that are difficult to identify or difficult to reach using a typical biopsy, the RTCs captured from the aphaeretic biopsy system can be used to identify such cancer clusters.

### Aphaeretic Biopsy System Overview

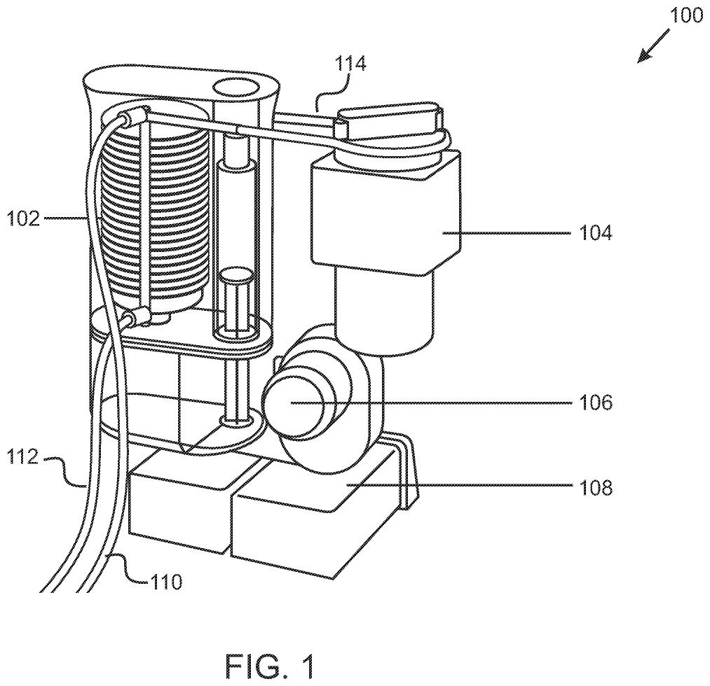

FIG. 1 illustrates an example aphaeretic biopsy system 100. As shown in FIG. 1, the aphaeretic biopsy system 100 can include a cartridge 102, a peristaltic pump 104, a syringe pump 106, and battery cells 108. Furthermore, a system can include a draw line 110 directing blood into the cartridge 102, and a return line 112. In some instances, the system as described herein can be utilized therapeutically and, in some cases, to debulk the tumor burden in the body.

A cartridge 102 can obtain a stream of blood (or a stream of blood cells) directly from a patient via a draw line 110 or from a container of blood. Blood can be drawn into the cartridge by a peristaltic pump 104 as the blood is directed along a peristaltic loop 114. Furthermore, in some instances, the blood can be combined with an anti-coagulant (as provided in syringe 106) to reduce clotting of cells prior to being directed to one or more microfluidic devices in a cartridge. In some instances, multiple draw lines and/or return lines can be implemented (e.g., as part of a multi-lumen catheter type system). A return line can be return blood directly to a patient or to a container.

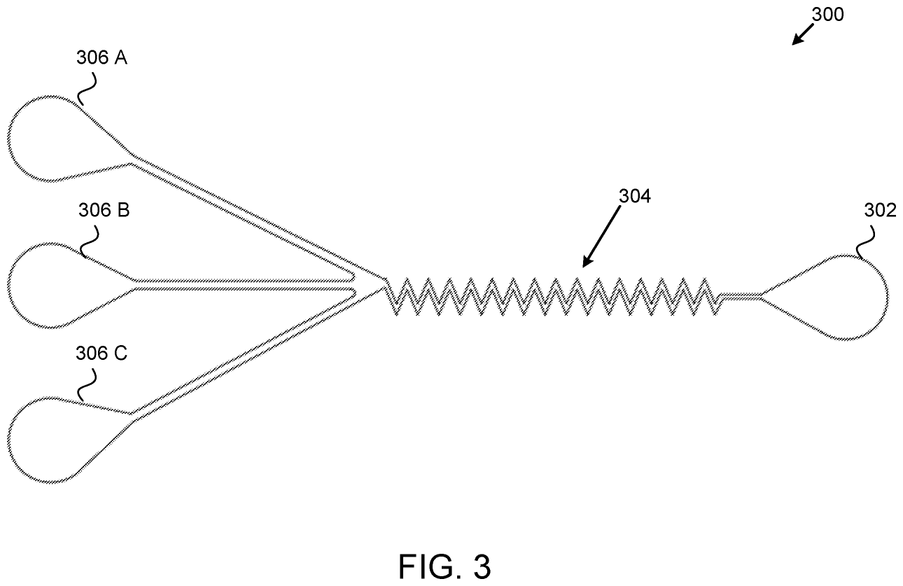

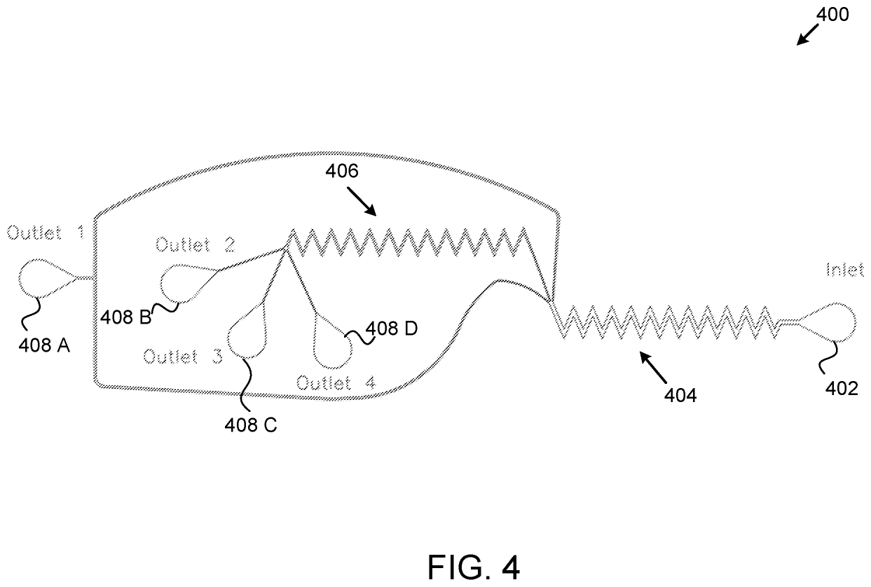

As described in greater detail below, a cartridge 102 can include one or more microfluidic devices configured to separate particles in the blood stream. For example, a microfluidic device can include multiple microfluidic channels incorporating inertial separation to separate RTCs from the bloodstream. The RTCs can be directed to a first outlet to be captured in a reservoir. The remaining portion of the blood stream can be directed to a second outlet to be returned to the patient via a return line 112. Example microfluidic devices are described in greater detail with respect to FIGS. 3-4 herein.

A syringe pump 106 can add an anti-coagulant into the blood stream using a rack and pinion system or other suitable system. Furthermore, battery cells 108 can be swappable and be used to provide electrical energy to the components of the aphaeretic biopsy system 100 as described herein.

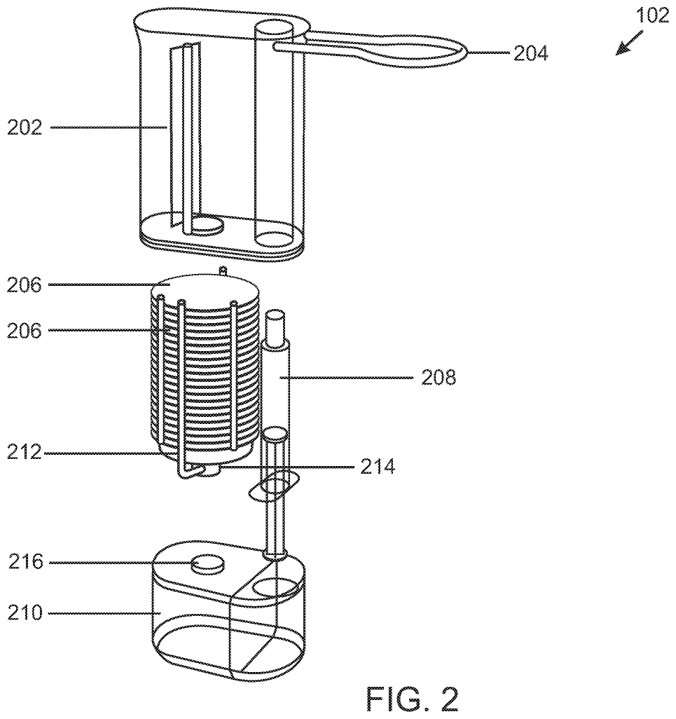

FIG. 2 is an exploded view of an example cartridge 102. The cartridge 102 can include a main housing 202 configured to hold the components in the cartridge 102. A peristaltic loop 204 can be connected to a peristaltic pump (e.g., 106 in FIG. 1) and configured to pressurize the stream of blood by the pump.

Furthermore, a cartridge 102 can include one or more microfluidic devices 206 (e.g. 1, 2, 3, 4, 5, 6, 7, 8, 9, 10, 15, 20, 25, 30, 40 or more), which can also be referred to as a stack 206. A microfluidic device comprises a set of micro-channels (microliter sized channels) etched or molded into a material (glass, silicon, or polymer such as PDMS). The micro-channels forming the microfluidic device are connected together to achieve the desired features (e.g., pumping and sorting). The stack 206 of microfluidic devices can be configured to each obtain a portion of the blood stream to each independently separate RTCs in respective portions of the bloodstream as described herein. For example, each microfluidic device in the stack 206 can include a first outlet configured to capture RTC cells (e.g., at return 214 to opening 216), and a second outlet to return the remaining portion of blood to the patient via a return line (via return 212). The captured RTC cells can be received at a reservoir 210. The devices in the stack 206 can be stacked vertically and/or horizontally, or any other suitable position.

The stack 206 can include a series of interconnected microfluidic devices. Each inertial microfluidic device can be used to separate particles, e.g., cells, by size and/or a flow rate. Despite such flow patterns not being fully understood, the flow patterns can be characterized by a set of equations: Reynold's Number, Particle Reynold's Number, Combined Lift Force, and Dean's Number. Reynold's Number

$$R e = \frac{ρ u L}{μ}$$

can indicate the laminarity of the flow because it is the ration of inertial to viscous forces. If the inertial forces outpower the viscous forces (Re>2000), then mixing can occur and particles may not focus to predictable streamlines.

Because particles of different sizes experience inertial and viscous forces differently, particle Reynolds number

$$R {e}_{p} = Re \times {( \frac{{a}_{p}}{L} )}^{2}$$

can also be used to characterize the ability of particles to focus due to inertial forces. The equation that best characterizes inertial focusing is the combined lift force

$${F}_{L} = \frac{ρ {U}_{m}^{2} {a}_{p}^{4}}{{D}_{h}^{2}} {C}_{L}$$

which can consider both flow characteristics and particle information. Although the combined lift force is accurate for Newtonian fluids, it can include an experimentally determined lift coefficient which varies based on the system and it assumes rigid, relatively neutrally buoyant particles. When curvature is present in the system, the Dean's number

$$D e = R e \sqrt{\frac{L}{2 {R}_{C}}}$$

can also be important as it characterizes the flow patterns that occur in curved systems.

A table depicting variables used in the set of equations is provided below.

TABLE 1

| Symbol | Variable |
|---|---|
| Re | Reynolds Number |
| ρ | Density (kg/m³) |
| u | Velocity (m/s) |
| L | Characteristic Channel Length (m) |
| μ | Dynamic Viscosity (kg/m s) |
| Reₚ | Particle Reynolds Number |
| aₚ | Particle Diameter (m) |
| F_{L} | Lift Force(kg m/s²) |
| Uₘ | Maximum Velocity (m/s) |
| Dₕ | Smallest Channel Dimension (m) |
| C_{L} | Lift Coefficient |
| De | Dean Number |
| R_{C} | Radius of Curvature (m) |

The study of these parameters has led to the development of spiral and serpentine devices to focus particles for a variety of applications, such as separating cells by size. The present aspects relate to a resistor-type device to focus particles of interest (e.g., cells) to separate streamlines from the other particles in the starting solution.

In many cases, directions have ranged from soft curves with large radii of curvature to right angles. However, such devices can generally not be used that have an angle sharper than 90°. The device as described herein can use sharp or relaxed angles (i.e., angles below 90° (e.g., 89, 80, 85, 75, 70, 65, 60, 55, 50, 45, 40, 35, 30, 25, 20° or less) or above 90° (e.g., 91, 95, 100, 110, 120, 130, 140, 150, 160, 170° or more) to strengthen the inertial separation. Furthermore, the device can include repeating 45° angles (e.g., 30 or more repeating angles (see e.g., FIG. 3) or 3, 4, 5, 10, 15, 20, 30, 40, 50, 60 or more repeating angles) to demonstrate the ability to achieve inertial separation for a distribution of particle sizes across a large range of flow rates. Other aspects of the device and systems can utilize obtuse angles in the shape of a trapezoid to achieve particle separation.

A design of a microfluidic device can include various aspects. For instance, a microfluidic device can provide single-stage sorting inertial separation devices. Furthermore, a channel in a microfluidic device can incorporate a sawtooth style design, a soft-edge resistor design, and/or a trapezoidal design to separate particles flowing through the channel in the microfluidic device. See PCT/US23/74932, and priority document U.S. Ser. No. 63/408,963, both entitled “SAWTOOTH INERTIAL DEVICE,” which are incorporated by reference herein in their entireties.

A microfluidic device used for particle separation can include an inlet for receiving particles of varying sizes across varying flow rates. The device can also include a main channel comprising a first end and a second end. The first end can be connected to the inlet. The main channel can comprise a series of angled portions. Each of the angled portions can form an angle that can be acute or obtuse (e.g., comprising angles between 1-89 degrees, or between 91 and 179 degrees) relative to an adjacent angled portion. The main channel can be configured to provide an inertial separation of the particles received at the inlet. The series of angled portions can reduce a pressure in the particle separation device and control varying flow rates of particles received at the inlet.

FIG. 3 illustrates an example particle separation device 300 with a sawtooth design. As shown in FIG. 3, the device 300 can include an inlet 302, a main channel 304, and multiple outlets 306A-C. Three outlets are shown, but 2, 3, 4, 5, or more outlets can be used. In an example, particles of varying sizes can be provided at the inlet 302, travel through the main channel and be output at outlets 306A-C. As an illustrative example, RTCs or other cells of interest can be directed to a first outlet 306A (and subsequently returned to a reservoir), while other particles in the blood stream can be directed to outlets 306B-C (and subsequently returned to the patient via a return line). The main channel can include a series of angled portions to provide inertial separation of particles based on a flow rate or a size of the particles, for example.

FIG. 4 illustrates an example particle separation device 400 with a double stage sawtooth design with a closed end outlet disposed between stages. For example, multiple stages 404, 406 of the main channel can be provided as a double stage resistor design with a closed end. Furthermore, as shown in FIG. 4, a closed end outlet (e.g., 408A) can be connected to the main channel between the stages 404, 406. Further, outlets 408B-D can be connected to a second stage 406 of the main channel.

Multiple microfluidic devices can be connected to independently separate particles received at the draw line. Multiple channels can direct blood from an input to separate microfluidic devices. In some instances, a first microfluidic device can separate some RTCs from a blood stream, directing a remaining portion of the bloodstream to a second microfluidic device. The second microfluidic device, in turn, can further separate any remaining RTCs in the blood stream, directing a further remaining portion of the bloodstream to the return line back to a patient.

In an aspect, an inlet or draw line can be attached to container comprising a blood sample. In another aspect, an inlet or draw line can directly attached to a patient via, for example, an arteriovenous fistula or graft, a hemodialysis catheter, a peritoneal catheter, or other appropriate needle or catheter. In an aspect a return line is attached to a container. In another aspect, a return line is attached directly to a patient via for example, an arteriovenous fistula or graft, a hemodialysis catheter, a peritoneal catheter, or other appropriate needle or catheter.

### Pressure Balancing Overview

In many cases, apheresis techniques can incorporate any of centrifugation, filtration, or adsorption to remove particles of interest from blood, all of which are designed to work with large sample volumes. Generally, techniques such as microfluidics have not been utilized in such applications, because they are designed to work with small sample volumes, which can mean they would need to be heavily multiplexed to be useful in applications such as apheresis.

To allow such systems to be multiplexed evenly such that they can be used in aphaeretic applications, the inlets may need to be properly pressure balanced to ensure each instance of the system is receiving an appropriate volume to operate within preferred specifications. Each of the channels of a device and/or a microfluidic chip has a length and a width that can be adjusted to ensure the pressure in each channel is as close to equal as possible as the stream of fluid flows through them. That is, each of the pressures in each channel can vary by about 30, 20, 10, 5, 1% or less.

In some instances, a series of particle separation devices are provided with pressure balancing. Pressure balancing multiple microfluidic devices can allow for even distribution of a fluid, such as a bloodstream across each microfluidic device, allowing for more efficient and an increased performance in separating particles from a fluid such as RTCs from a bloodstream.

Pressure balancing can be accomplished with either an active or passive system. Active systems generally may require more parts and therefore look more complex, but they also may be more reliable and with a simpler design. Passive systems take advantage of the pressure difference in various sized channels to balance the pressure going into each instance of the system.

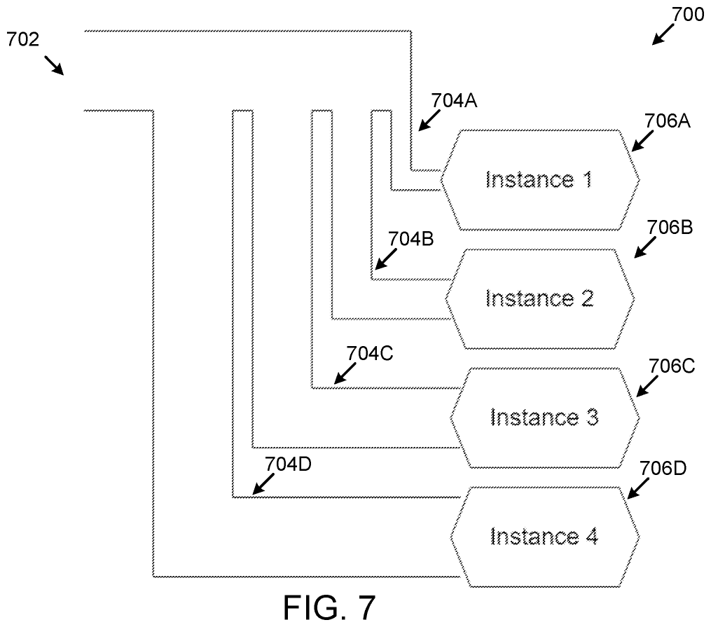

In a first example aspect, a particle separation system comprising multiple microfluidic devices with a balanced pressure across the multiple microfluidic devices is provided. The system can include an inlet (e.g., 702 in FIG. 7) obtaining a stream of blood cells. The system can also include a plurality of microfluidic devices (e.g., 706A-D in FIG. 7) each configured to obtain a portion of the stream of blood cells from the inlet. Four microfluidic devices are shown, but any number can be used (e.g., 2, 5, 10, 20, 30, 40, 50, or more). Balanced pressure across the multiple microfluidic devices means that the pressure of the fluid entering each microfluidic device in the stack is about the same, e.g., differing by less than 20, 25, 10, 5, 3, 2, 1% or less.

Each of the plurality of microfluidic devices include at least one separation channel (e.g., 304 in FIG. 3, 404 in FIG. 4) to separate particles in the stream of blood cells. Each of the plurality of microfluidic devices can also include at least a first outlet for capturing released tumor cells included in the stream of blood cells and a second outlet for returning a remaining portion of the blood cells via a return line.

The system can also include a series of channels (e.g., 704A-D in FIG. 7) connecting the inlet to each of the plurality of microfluidic devices. Four channels are shown, but any number can be used (e.g., 2, 3, 4, 5, or more). Each of the channels can include a length and a width that is variable to balance a pressure of each of the channels as the stream of blood cells are directed through each channel. For instance, the series of channels include at least a first channel connecting the inlet to a first microfluidic device, with the first channel comprising a first length and width. The series of channels can also include a second channel connecting the inlet to a second microfluidic device, with the second channel comprising a second length and width greater than that of the first channel. While the length and widths can be variable, in an aspect a channel length can be about 1 mm, 5 mm, 10 mm, 20 mm, 30 mm, 50 mm, 75 mm, 100 mm, 2 cm, 3 cm, 4 cm, 5 cm, 6 cm, 7 cm, 8 cm, 9 cm, 10 cm or more and can have a width of about 1 μm, 5 μm, 10 μm, 100 μm, 250 μm, 500 μm, 750 μm, 1 mm, 2 mm, 3 mm, 4 mm, 5 mm or more.

In some instances, the length and width of each of the series of channels is based on a major pressure loss due to friction loss as each portion of the stream of blood cells travel through each channel when all flow is laminar. In some instances, determining the major pressure loss is based on a dynamic viscosity, the length, a volumetric flow rate, and a cross sectional area of each of the series of channels. In some instances, the length and width of each of the series of channels is based on a fluid density, a hydraulic diameter, a mean flow velocity, and a flow coefficient of each of the series of channels.

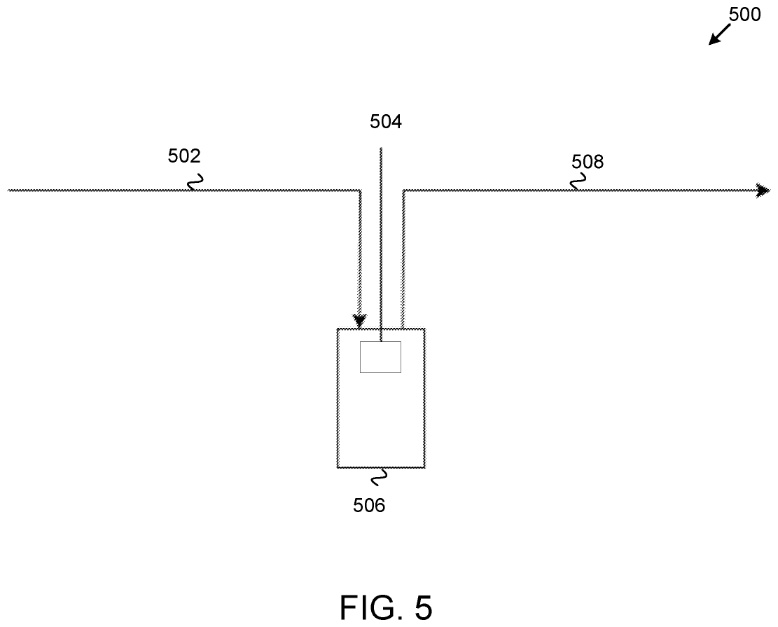

In some instances, the system can include any of a valve, a chamber, and/or a pump (e.g., 504 in FIG. 5) connected to each channel. The valve, chamber, and/or pump are configured to modify and control the pressure of each channel. In some instances, the system can include a closed-loop control system configured to adjust the pressure of the pump based on detected pressure changes in any of the channels. In some instances, the system can include a reservoir pump connected to a reservoir configured to capture the released tumor cells output from the first output, wherein the reservoir is configured to be pressurized.

In another example aspect, a device is provided that can include an inlet obtaining a stream of blood cells. A device can also include a plurality of microfluidic devices (e.g., 2, 5, 10, 15, 20, 30, 40, 50, or more) each configured to obtain a portion of the stream of blood cells from the inlet.

Each of the plurality of microfluidic devices can include at least one separation channel to separate particles in the stream of blood cells, at least a first outlet for capturing released tumor cells included in the stream of blood cells, and a second outlet for returning a remaining portion of the blood cells via a return line.

The device can also include a series of channels connecting the inlet to each of the plurality of microfluidic devices. Each of the channels includes a length and a width that is variable to balance a pressure of each of the channels as the stream of blood cells are directed through each channel. The device can also include any of a valve, a chamber, and/or a pump connected to each channel. The valve, chamber, and/or pump can be configured to modify and control the pressure of each channel.

In some instances, the device can include a closed-loop control system configured to adjust the pressure of the pump based on detected pressure changes in any of the channels. In some instances, the device can include a pump connected to a reservoir configured to capture the released tumor cells output from the first output, wherein the reservoir is configured to be pressurized.

In some instances, the length and width of each of the series of channels is based on a major pressure loss due to friction loss as each portion of the stream of blood cells travel through each channel when all flow is laminar. In some instances, the length and width of each of the series of channels is based on a major pressure loss due to friction loss as each portion of the stream of blood cells travel through each channel when all flow is laminar. In some instances, the length and width of each of the series of channels is based on a fluid density, a hydraulic diameter, a mean flow velocity, and a flow coefficient of each of the series of channels.

FIG. 5 illustrates an example active pressure balancing system 500. For example, in FIG. 5, a draw line 502 from a patient can be input into a chamber 506 comprising a pump 504. The pump 504 can pressurize the bloodstream input from the draw line 502. The blood input from the draw line 502 can be separated into multiple chambers, with a first chamber 506 obtaining a portion of the blood stream. Multiple chambers can independently pressurize portions of the blood stream using one or more pumps. Pressurizing blood included in each chamber can balance the pressure across each channel, allowing for increased performance in separating RTCs in each microfluidic device as described herein.

After pressurizing the blood in a chamber 506, the blood can be directed to the aphaeretic system via channel 508. For instance, the pressurized blood can be directed to one or more microfluidic devices as described herein.

Active systems have a valve, chamber, pump, or similar device on each line to set the pressure going into each instance of the system. Active systems can potentially involve a closed-loop control to ensure constant flow rate passing through the system. For example, an active system can have the blood drawn split into multiple chambers, each of which goes into a pressurized chamber that then is drawn up into a single instance of the system. In the example, a closed-loop control can adjust the pressure of the pressure pump in accordance with the pressure changes in the line from the patient to ensure constant flow rate going into the aphaeretic system.

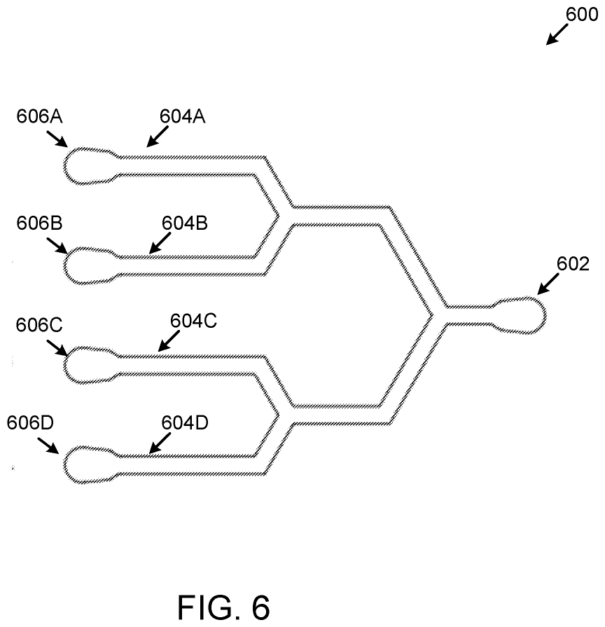

In other instances, a passive pressure balancing system can balance the pressure across the aphaeretic system. FIG. 6 illustrates a first example passive pressure balancing system 600. For example, in FIG. 6, an inlet 602 can split portions of blood across channels 604A-D to outlets 606A-D. Four channels and four outlets are shown but any number of channels and outlets can be used (e.g. 1, 2, 3, 4, 5, or more). Furthermore, as described in greater detail below, the length and width of each channel 604A-D can be modified to balance a pressure of each channel 604A-D to allow for equivalent blood distribution across each channel 604A-D.

In general, passive pressure balancing can be achieved by tuning the major loss and minor loss involved in a flow device. The major loss refers to the pressure loss due to the friction loss as the flow travels in the channel. The major loss can be adjusted by changing the length and width of flow channels. The minor loss refers to the flow loss due to the energy losses associated with flow separation. Examples of minor loss include but are not limited to the losses associated with flow channel contraction/expansion, flow channel changing direction, flow through valves or other devices. The simplest version of passive pressure balancing ensures that each line is identical to all other lines. For example, FIG. 6 demonstrates a microfluidic version of this pressure balancing method. Tubing can also be used to achieve the same goal by making all tubing lengths and connectors identical.

In many instances, the lengths and widths of each channel can be modified to achieve a more balanced pressure across the channels. FIG. 7 illustrates an example passive pressure balancing system 700 with varied lengths and widths of each channel. For example, in FIG. 7, from an inlet 702, a first channel 704A can include a lesser length and width than channels 704B-D. Four channels are shown, but any number can be used (e.g., 1, 2, 3, 4, 5, or more). Each channel 704A-D can direct portions of the blood stream into corresponding instances 706A-D (such as different microfluidic devices as described herein).

Channels can be passively pressure balanced by changing the length and width of the lines to each instance of the system such that they all have the same pressure. This is particularly useful when the number of instances is high. For example, FIG. 7 illustrates one example of this where longer lines are wider to decrease the pressure drop due to the longer lines. Other pressure changing mechanisms can be used as well, such as contracting portions of channels or lines and/or adding one or more (e.g., 1, 2, 3, 4, 5, 10, 20 or more) sharp angles (less than 90° angles, e.g. 10, 20, 30, 40, 45, 50, 60, 70, or) 80°.

Assuming all flow is laminar, the Hagen-Poiseuille law (equation 1) can be used to calculate the major pressure drop on each line such that they can be manipulated to be balanced. This equation is dependent on the dynamic viscosity (μ), length (L), volumetric flow rate (Q), and cross-sectional area of the line (A). In this system, all lines contain the same fluid so the dynamic viscosity will be identical and the volumetric flow rate into each device should be the same. By setting Δp equal for each line, equation 2 is obtained. To properly pressure balance this system, the length and cross-sectional area are varied appropriately for this equation to hold. In addition to the pressure loss due to the length and area of the pipe, minor losses also occur anytime the line does something other than flow straight such as expand/contract or change direction. The pressure drop due to common additions to flow lines such as right angles can be estimated from look-up tables, but an exact pressure drop may need to be determined experimentally.

$$Δ p = \frac{8 πμ LQ}{{A}^{2}} \qquad Equation ( 1 )$$

$$\frac{{L}_{1}}{{A}_{1}^{2}} = \frac{{L}_{2}}{{A}_{2}^{2}} \qquad Equation ( 2 )$$

If non-laminar flow is present, the more general Darcy-Weisbach equation (equation 3) can be used where ρ is the fluid density, D_{H} is the hydraulic diameter, [symbol] v [symbol] is the mean flow velocity, and λ is the flow coefficient. Making the appropriate substitution of A for laminar flow yields the Hagen-Poiseuille equation. Estimations for λ depend on the exact system characteristics such as laminar vs turbulent flow and material roughness.

$$Δ p = λ \frac{ρ {〈 v 〉}^{2} L}{2 {D}_{H}} \qquad Equation ( 3 )$$

In some instances, a collection reservoir can be pressurized using a pump. For instance, the outlets of the microfluidic chips can be pressure balanced to a collection reservoir. The reservoir itself can be pressurized to mimic the biological system (e.g., human circulatory system).

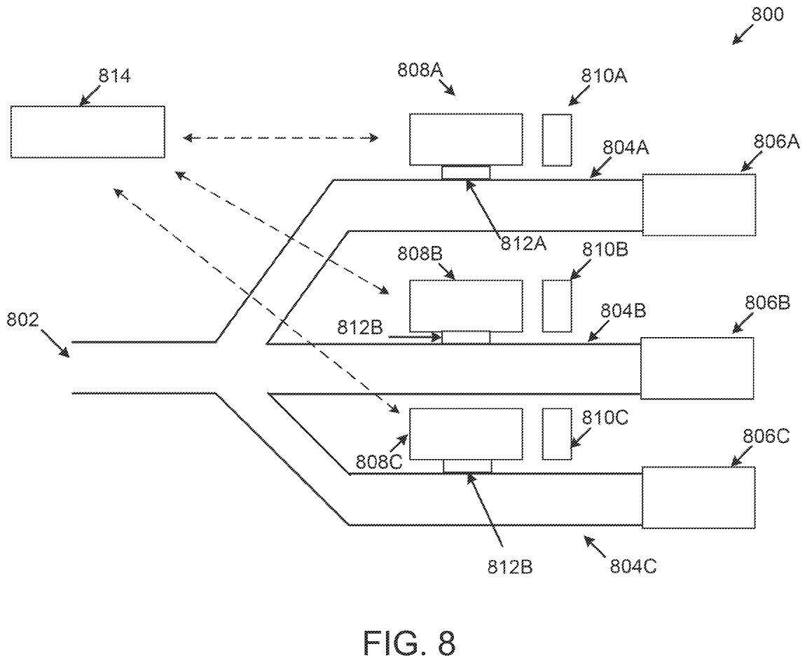

FIG. 8 illustrates an example series of microfluidic devices with an active pressure balancing system 800. As shown in FIG. 8, the system 800 can include an inlet 802 and a series of channels 804A-C splitting off of the inlet 802. Three channels are shown, but any number can be used (e.g., 1, 2, 3, 4, 5, or more). A flow of blood is configured to flow from the inlet 802, through each channel 804A-C to corresponding microfluidic devices 806A-C. Devices 806A-C can include any type of device capable of separating particles, such as microfluidic devices as described herein (e.g., 300 in FIG. 3, 400 in FIG. 4).

As described herein, each channel can include any of a valve, chamber, or a pump. In some instances, a chamber can be formed in each channel and pressurized via a pump 808A-C connected to each channel. Three pumps are shown, but any number can be used (e.g., 1, 2, 3, 4, 5, or more). Each pump 808A-C can be connected to each channel 804A-C (or a chamber formed in each channel) via a valve 812A-C. The pumps 808A-C can pressurize each channel 804A-C so as to pressure balance each channel as described herein.

In some instances, a portion of the RTCs are configured to be held in a first chamber (e.g., a chamber in channel 804A) that are pressurized by a first pump (e.g., pump 808A. The closed-loop control system 814 can be configured to obtain a pressure level by a pressure sensor (e.g., 810A) and instruct the first pump to modify the pressure level in the first chamber to maintain a specified pressure level.

Furthermore, a pressure sensor 810A-C can monitor the pressure provided by each pump 808A-C and/or a pressure of each chamber 804A-C. The pressure sensors 810A-C can feed pressure information to a closed-loop system 814 to automatically adjust the pressure of each channel 804A-C. For example, the closed loop system 814 can monitor each pressure reading provided by sensors 810A-C and instruct pumps 808A-C to modify the pressurizing of each channel 804A-C. For example, if a first channel 804A is below a specified level (as detected by sensor 810A), the closed loop system 814 can instruct the pump 808A to increase the pressure in the channel 804A. The closed loop system 814 can implement one or more computers to identify a pressure value for each channel, determine whether a corrective action is needed for a channel, and instruct a pump to modify the pressure of the channel. In some instances, the valves can be adjusted to modify the pressure in the channel.

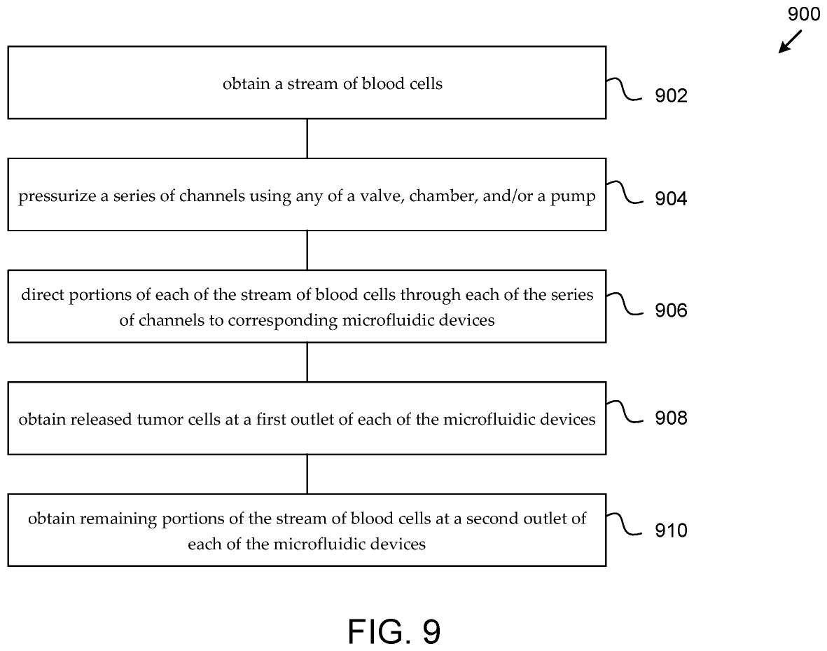

FIG. 9 is a flow process 900 of an example method for separating released tumor cells from a blood stream through a pressure balanced aphaeretic system. At 902, the method can include obtaining, at an inlet, a stream of blood cells.

At 904, the method can also include pressurizing a series of channels using any of a valve, chamber, and/or a pump. In some instances, the method can include determining a pressure in each of a series of channels connecting an inlet to a plurality of microfluidic devices and modifying a length and a width for each of the series of channels based on the pressure in each of a series of channels.

At 906, the method can also include directing portions of each of the stream of blood cells through each of the series of channels to corresponding microfluidic devices. At least one separation channel for each microfluidic device can be configured to separate particles in each portion of the stream of blood cells.

At 908, the method can also include obtaining released tumor cells at a first outlet of each of the microfluidic devices. In some instances, the method can include pressurizing a reservoir using a reservoir pump and capturing the released tumor cells from the first outlet at the reservoir.

At 910, the method can also include obtaining remaining portions of the stream of blood cells at a second outlet of each of the microfluidic devices.

In some instances, the length and width of each of the series of channels is based on a major pressure loss due to friction loss as each portion of the stream of blood cells travel through each channel when all flow is laminar. In some instances, determining the major pressure loss is based on a dynamic viscosity, the length, a volumetric flow rate, and a cross sectional area of each of the series of channels. In some instances, the length and width of each of the series of channels is based on a fluid density, a hydraulic diameter, a mean flow velocity, and a flow coefficient of each of the series of channels.

### Removable Cartridge Overview

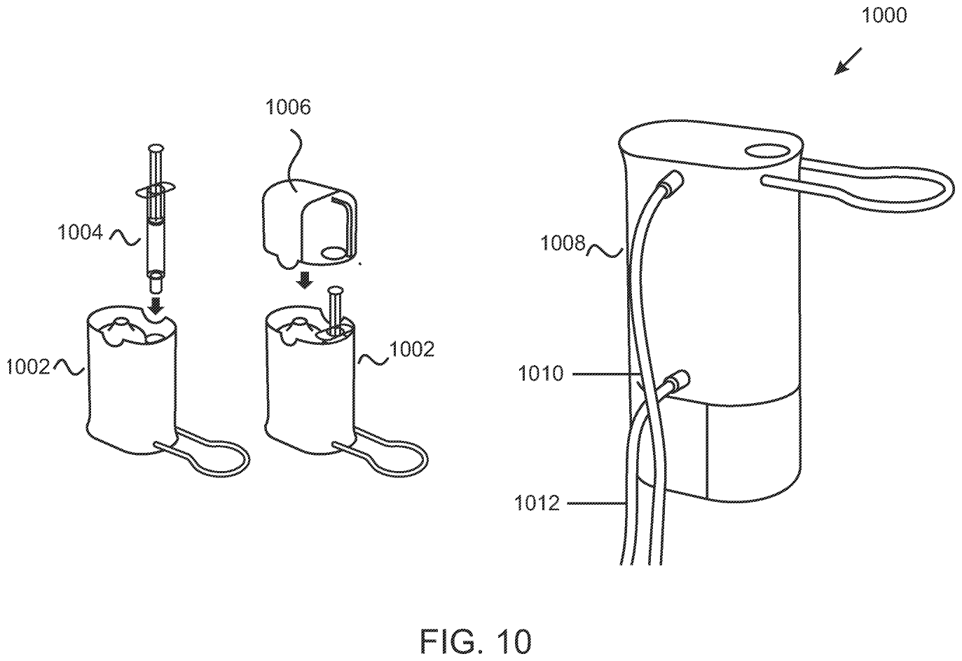

As described above, an aphaeretic biopsy system can include a removable cartridge. A cartridge can be added to an outer housing and can be used to separate RTCs from a bloodstream. FIG. 10 illustrates an example cartridge 1000. As shown in FIG. 10, the cartridge can include a housing 1002. The housing 1002 can contain a syringe 1004, 208 comprising an anti-coagulant, such as heparin, sodium citrate, or acid citrate dextrose, for example. Furthermore, the housing 1002 can be connected to a reservoir 1006 configured to capture RTCs separated from the bloodstream.

Furthermore, the assembled cartridge housing 1008 can include a draw line 1010 providing blood to the cartridge and a return line 1012 returning blood (without RTCs) to the patient.

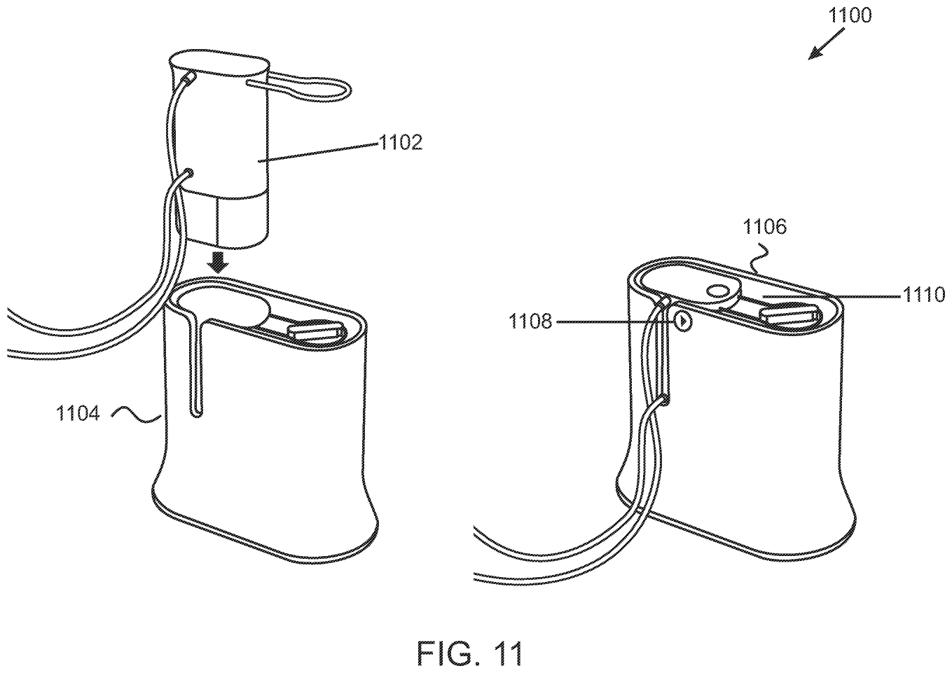

The cartridge can be removably engaged to the outer housing. For instance, after one or more uses, the cartridge can be removed from the housing and a new cartridge can be replaced for subsequent use. FIG. 11 illustrates the inserting of a cartridge in an outer housing as part of an aphaeretic biopsy system 1100. As shown in FIG. 11, a cartridge 1102 can be inserted into a cavity of the outer housing 1104. Once inserted, the cartridge 1102 can reside within the outer housing 1004.

The outer housing 1104 can include an outer shell, with one or more control interfaces (e.g., 1108) on the outer housing. For example, a play button can be added to the outer housing to start a dialysis process. A cap 1106 (such as a metallic or plastic cap) can be disposed on a top surface of the outer housing. Furthermore, the draw line (or peristaltic loop 1110) can be directed around a pump disposed within the outer housing.

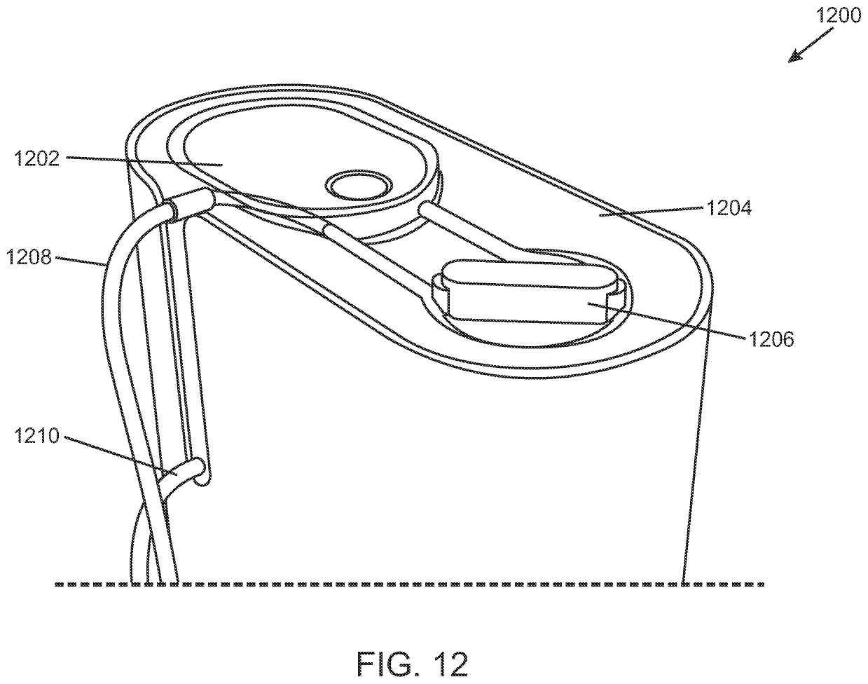

FIG. 12 illustrates a top view of an aphaeretic biopsy system 1200. As shown in FIG. 12, a cartridge 1202 can be disposed within the housing. Furthermore, a cap 1204 (such as a metallic or plastic cap) can enclose a top portion of the outer housing. A peristaltic loop 1206 can direct the bloodstream to a pump to pressurize the bloodstream. Additionally, a draw line 1208 can provide blood to the cartridge and a return line 1210 can return the remaining blood to the patient.

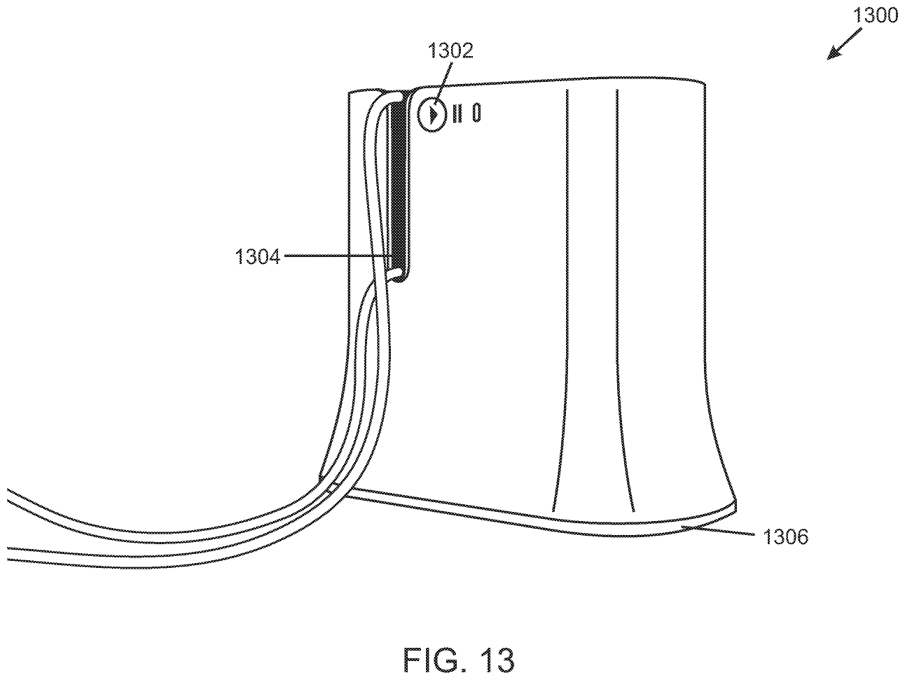

FIG. 13 illustrates a side view of an example aphaeretic biopsy system 1300. As shown in FIG. 13, a number of interfaces and indicators (e.g., 1302) can be added to the outer housing 1304. For example, controls to start, cancel, pause the dialysis system. Furthermore, indicators relating to a status of the system, a low battery warning, a time remaining indicator, and/or a pairing indicator can be included. In some instances, a system 1300 can be wirelessly controlled to control the operation of the system. In some instances, a system 1300 can include sensors for monitoring air bubbles, clots, and a pressure in the draw line and/or the return line. The outer housing can include a frosted surface and the surface can be vacuum metallized. Furthermore, the housing can include a base 1306 (such as a metallic or plastic base).

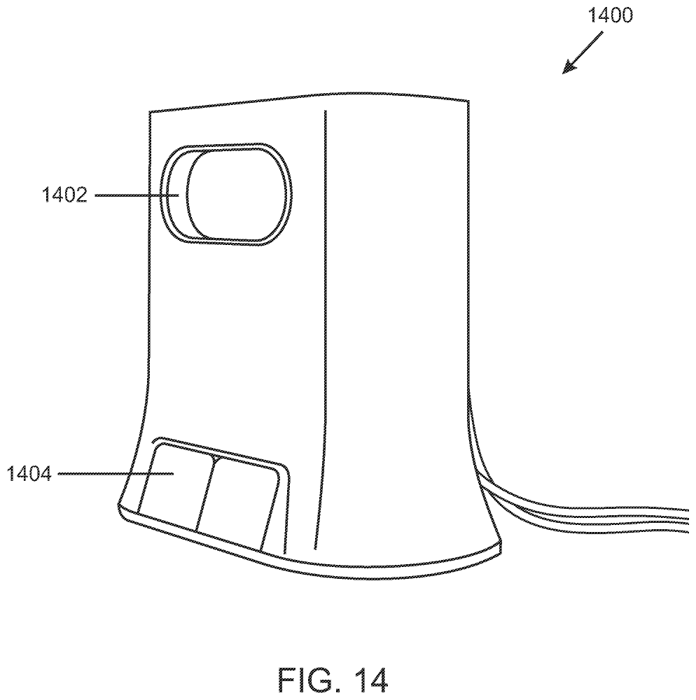

FIG. 14 illustrates a rear view of an example aphaeretic biopsy system 1400. As shown in FIG. 14, the rear portion of the outer housing can include a handle 1402 to move the system. Furthermore, an opening 1404 can be used to add or replace batteries in the outer housing, allowing for easy replacement of the batteries. Power can also be supplied by a wired connection.

In an example aspect, an aphaeretic biopsy system is provided. An aphaeretic biopsy system can obtain a stream of blood cells, separate RTCs from the blood stream, and return a remaining portion of the stream of blood cells to a patient. The aphaeretic biopsy system can include an outer housing (e.g., 1104 in FIG. 11), a peristaltic pump (e.g., 104 in FIG. 1) within the outer housing, and a removable cartridge (e.g., 102 in FIG. 1) configured to be disposed within the outer housing.

The removable cartridge can include a draw line (e.g., 110 in FIG. 1) configured to direct a stream of blood cells into the removable cartridge. Furthermore, a peristaltic pump (e.g., 104) can be configured to pressurize the stream of blood cells by directing the stream of blood cells around a peristaltic loop (e.g., 114 in FIG. 1). In some instances, an aphaeretic biopsy system can include a syringe including an anti-coagulant configured to be added to the stream of blood cells.

The removable cartridge can also include one or more microfluidic devices (e.g., 206 in FIG. 2). The one or more microfluidic devices can be configured to separate cells in the stream of blood cells that are directed through the one or more microfluidic devices.

In some instances, the one or more microfluidic devices include at least one channel (e.g., 304 in FIG. 3, 404 in FIG. 4) that includes a series of angled portions to separate particles (e.g. RTCs) in the stream of blood cells. The at least one channel can be about 1, 2, 3, 4, 5, or more channels. Furthermore, in some instances, the one or more microfluidic devices include multiple microfluidic sorting channels (e.g., first channel 404, second channel 406 in FIG. 4). Multiple microfluidic sorting channels can be 1, 2, 3, 4, 5, or more channels. Each of the multiple microfluidic sorting channels can include corresponding series of angled portions (e.g., 1, 2, 3, 4, 5, 10, 20, 30, 40 or more) to sort particles in the stream of blood cells by a size and/or a flow rate to direct cells into either the first outlet or the second outlet. In some instances, the cartridge comprises a stack comprising a plurality of connected microfluidic devices (e.g., stack 206 in FIG. 2). Each of the microfluidic devices in the stack can direct the stream of blood cells toward a first outlet, a second outlet, a third outlet, a fourth outlet, or a fifth outlet, etc.

A removable cartridge can also include a reservoir (e.g., 210 in FIG. 2) configured to capture RTCs or circulating tumor cells (CTC) cells obtained from, for example, a first outlet of the one or more microfluidic devices. The removable cartridge can also include a return line (e.g., 112 in FIG. 1) configured to direct a remaining portion of the stream of blood cells obtained from a second outlet of any of the one or more microfluidic devices. In some instances, the outer housing further includes a swappable battery pack (e.g., 108 in FIG. 1) and one or more controls (e.g., 1302 in FIG. 13) to control various aspects of the aphaeretic biopsy system.

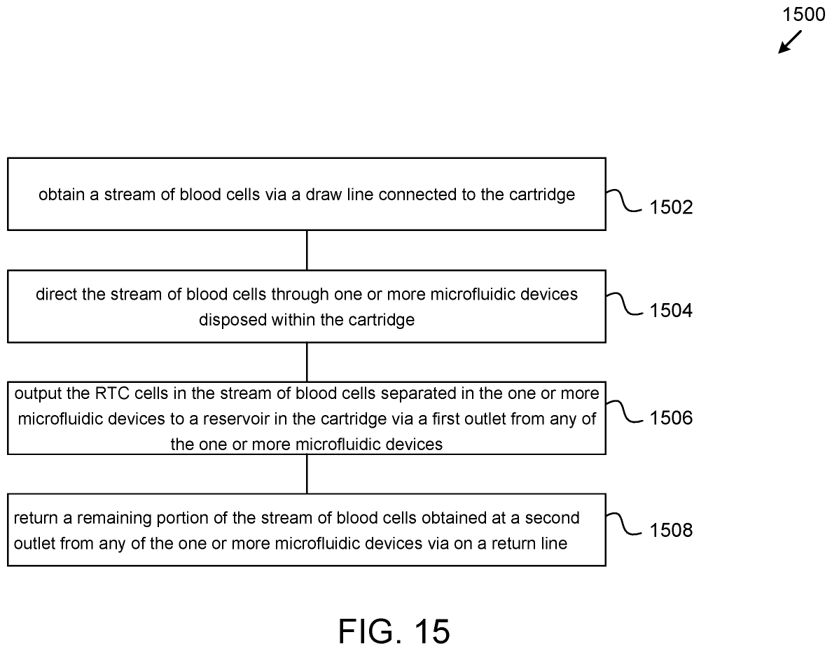

FIG. 15 illustrates an example method 1500 for configuring an aphaeretic biopsy system. At 1502, the method can include obtaining, at a cartridge, a stream of blood cells via a draw line connected to the cartridge. The stream of blood cells can be drawn into the removable cartridge via a peristaltic pump within the outer housing.

In some instances, prior to obtaining the blood, an amount of saline or another similar fluid can be drawn into the removable cartridge. The saline can be used to reduce the amount of air (and air bubbles) in the system. After the introduction of the saline, the return line can be added to the patient, and the blood can be drawn into the cartridge. The cartridge can include a valve or wall to switch between allowing saline and blood in the cartridge.

In some instances, pressuring the stream of blood cells 1504 includes directing the stream of blood cells along a peristaltic loop connecting the draw line to the cartridge, wherein the peristaltic loop is connected to the peristaltic pump.

In some instances, the method can include inserting a syringe including the anti-coagulant into the cartridge. The anti-coagulant can be added to the stream of blood cells. The method can also include installing the reservoir and the one or more microfluidic devices, and adding the draw line and the return line to the cartridge. In some instances, the cartridge is removable from an outer housing of the aphaeretic biopsy system.

At 1506, the method can include directing the stream of blood cells through one or more microfluidic devices disposed within the cartridge, wherein the one or more microfluidic devices are configured to separate cells in the stream of blood cells as the stream of blood cells are directed through the one or more microfluidic devices.

In some instances, the one or more microfluidic devices include at least one channel that includes a series of angled portions to separate particles in the stream of blood cells. In some instances, the one or more microfluidic devices include multiple microfluidic sorting channels, each of the multiple microfluidic sorting channel including corresponding series of angled portions to sort particles in the stream of blood cells by a size and/or a flow rate to direct cells into either a first outlet or a second outlet (or other outlet where used). In some instances, the cartridge comprises a stack comprising a plurality of connected microfluidic devices. Each of the microfluidic devices in the stack direct the stream of blood cells can be directed toward either a first outlet or a second outlet (or other outlet where used).

At 1508, the method can include outputting the CTC cells in the stream of blood cells separated in the one or more microfluidic devices to a reservoir in the cartridge via a first outlet from any of the one or more microfluidic devices.

At 1510, the method can include returning a remaining portion of the stream of blood cells obtained at a second outlet from any of the one or more microfluidic devices via on a return line.

In an aspect a system can process a broad range of blood volumes (i.e., processing throughput). In an aspect a microfluidic chip of a system described herein can process a biological fluid (e.g., blood) volume from about ranging from 5, 10, 15, 20, 30, 40, 50, 100, 200, 500, 1,000, 2,000, 3,000, 4,000, 5,000 mL or more at a throughput of about 10, 50, 100, 250, 500, 750, 1,000, 2,000, 3,000, 4,000, 5,000 UL or more per min per microfluidic chip. In an aspect a microfluidic chip can process a biological fluid at a throughput of about 500 μL to about 3 mL/min per microfluid chip. Therefore, depending on the number of microfluidic chips used, a biological fluid can be processed in a few minutes (e.g., 60, 45, 30, 20, 10, 5 minutes or less) or a few hours (5, 4, 3, 2, 1 or less hours).

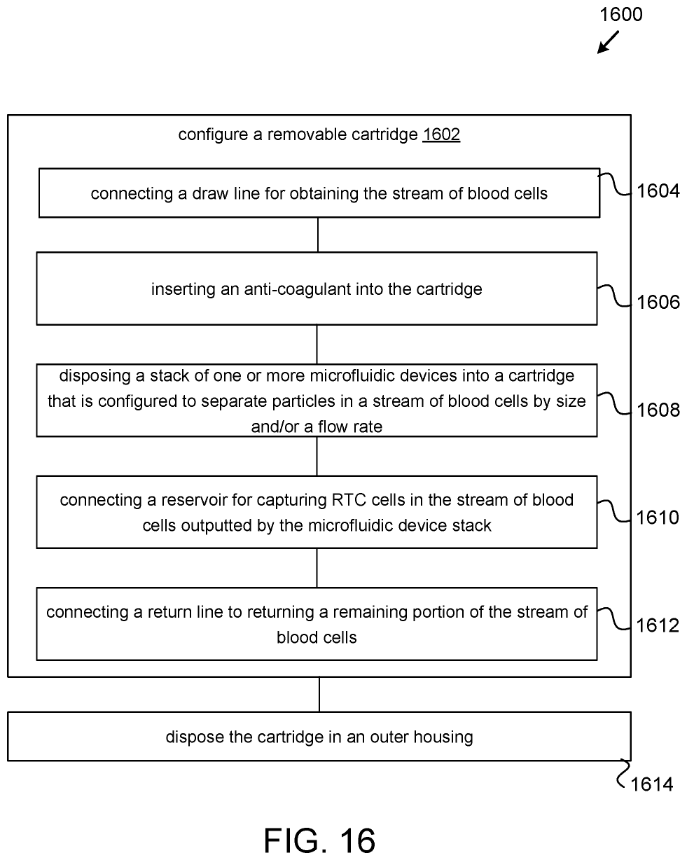

FIG. 16 illustrates an example method for configuring an aphaeretic biopsy system. At 1602, the method can include configuring a removable cartridge.

At 1604, configuring the removable cartridge can include connecting a draw line to a cartridge. The draw line can be configured to direct a stream of blood cells to the cartridge. In some instances, the method can include connecting a peristaltic loop connecting the cartridge to a peristaltic pump. The stream of blood cells can be configured to be pressurized by the peristaltic pump prior to being provided to the stack of one or more microfluidic devices.

At 1606, configuring the removable cartridge can include inserting a syringe comprising an anti-coagulant into the cartridge. The anti-coagulant can be configured to be added to the stream of blood cells.

At 1608, configuring the removable cartridge can include disposing a stack of one or more microfluidic devices into the cartridge. The stack of one or more microfluidic devices can be configured to separate particles in the stream of blood by a particle size and/or a flow rate.

In some instances, the one or more microfluidic devices include at least one channel that includes a series of angled portions to separate particles in the stream of blood cells. In some instances, the one or more microfluidic devices include multiple microfluidic sorting channels, each of the multiple microfluidic sorting channel including corresponding series of angled portions to sort particles in the stream of blood cells by a size and/or a flow rate to direct cells into either the first outlet or the second outlet.

At 1610, configuring the removable cartridge can include connecting a reservoir to the cartridge. The reservoir can be configured to capture RTCs or CTCs outputted at a first outlet of any of the one or more microfluidic devices.

At 1612, configuring the removable cartridge can include connecting a return line to the cartridge. The return line can be configured to direct a remaining portion of the stream of blood cells outputted at a second outlet of any of the one or more microfluidic devices.

At 1614, the method can include disposing the cartridge within an outer housing.

### Computer Device Overview

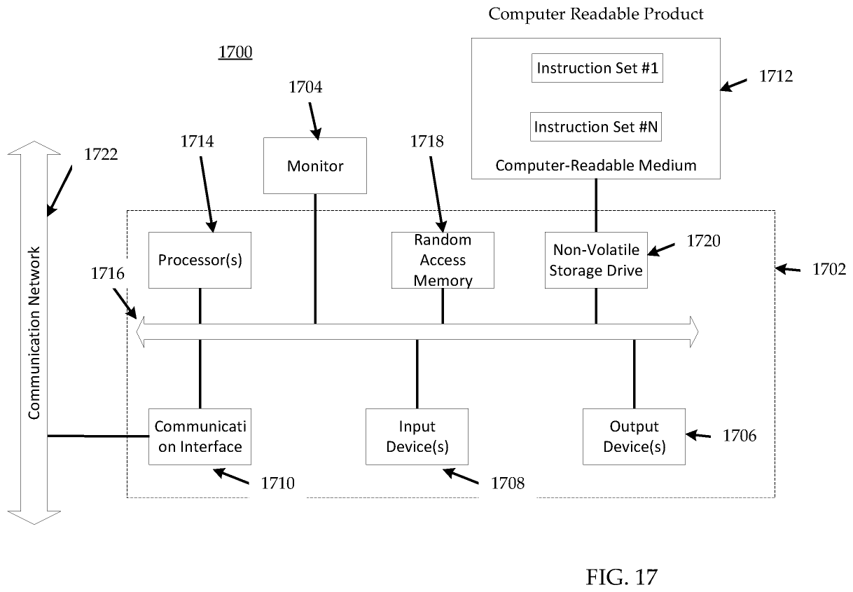

FIG. 17 is a block diagram of a special-purpose computer system 1700 according to an aspect. For example, system 1700 can deployed as part of a closed loop system 814 in FIG. 8 or part of a computer system disposed in a aphaeretic biopsy system as described herein. The methods and processes described herein can similarly be implemented by tangible, non-transitory computer readable storage mediums and/or computer-program products that direct a computer system to perform the actions of the methods and processes described herein. Each such computer-program product can comprise sets of instructions (e.g., codes) embodied on a computer-readable medium that directs the processor of a computer system to perform corresponding operations. The instructions can be configured to run in sequential order, or in parallel (such as under different processing threads), or in a combination thereof.

Special-purpose computer system 1700 comprises a computer 1702, a monitor 1704 coupled to computer 1702, one or more additional user output devices 1706 (optional) coupled to computer 1702, one or more user input devices 1708 (e.g., keyboard, mouse, track ball, touch screen) coupled to computer 1702, an optional communications interface 1710 coupled to computer 1702, and a computer-program product including a tangible computer-readable storage medium 1712 in or accessible to computer 1702. Instructions stored on computer-readable storage medium 1712 can direct system 1700 to perform the methods and processes described herein. Computer 1702 can include one or more processors 1714 that communicate with a number of peripheral devices via a bus subsystem 1716. These peripheral devices can include user output device(s) 1706, user input device(s) 1708, communications interface 1710, and a storage subsystem, such as random access memory (RAM) 1718 and non-volatile storage drive 1720 (e.g., disk drive, optical drive, solid state drive), which are forms of tangible computer-readable memory.

Computer-readable medium 1712 can be loaded into random access memory 1718, stored in non-volatile storage drive 1720, or otherwise accessible to one or more components of computer 1702. Each processor 1714 can comprise a microprocessor, such as a microprocessor from Intel® or Advanced Micro Devices, Inc.®, or the like. To support computer-readable medium 1712, the computer 1702 runs an operating system that handles the communications between computer-readable medium 1712 and the above-noted components, as well as the communications between the above-noted components in support of the computer-readable medium 1712. Exemplary operating systems include Windows® or the like from Microsoft Corporation, Solaris® from Sun Microsystems, LINUX, UNIX, and the like. In many aspects and as described herein, the computer-program product can be an apparatus (e.g., a hard drive including case, read/write head, etc., a computer disc including case, a memory card including connector, case, etc.) that includes a computer-readable medium (e.g., a disk, a memory chip, etc.). In other aspects, a computer-program product can comprise the instruction sets, or code modules, themselves, and be embodied on a computer-readable medium.

User input devices 1708 include all possible types of devices and mechanisms to input information to computer system 1702. These can include a keyboard, a keypad, a mouse, a scanner, a digital drawing pad, a touch screen incorporated into the display, audio input devices such as voice recognition systems, microphones, and other types of input devices. In various aspects, user input devices 1708 are typically embodied as a computer mouse, a trackball, a track pad, a joystick, wireless remote, a drawing tablet, a voice command system. User input devices 1708 typically allow a user to select objects, icons, text and the like that appear on the monitor 1704 via a command such as a click of a button or the like. User output devices 1706 include all possible types of devices and mechanisms to output information from computer 1702. These can include a display (e.g., monitor 1704), printers, non-visual displays such as audio output devices, etc.

Communications interface 1710 provides an interface to other communication networks and devices and can serve as an interface to receive data from and transmit data to other systems, WANs and/or the Internet, via a wired or wireless communication network 1722. In addition, communications interface 1710 can include an underwater radio for transmitting and receiving data in an underwater network. Aspects of communications interface 1710 typically include an Ethernet card, a modem (telephone, satellite, cable, ISDN), a (asynchronous) digital subscriber line (DSL) unit, a FireWire® interface, a USB® interface, a wireless network adapter, and the like. For example, communications interface 1710 can be coupled to a computer network, to a FireWire® bus, or the like. In other aspects, communications interface 1710 can be physically integrated on the motherboard of computer 1702, and/or can be a software program, or the like.

RAM 1718 and non-volatile storage drive 1720 are examples of tangible computer-readable media configured to store data such as computer-program product aspects of the present methods and devices, including executable computer code, human-readable code, or the like. Other types of tangible computer-readable media include floppy disks, removable hard disks, optical storage media such as CD-ROMs, DVDs, bar codes, semiconductor memories such as flash memories, read-only-memories (ROMs), battery-backed volatile memories, networked storage devices, and the like. RAM 1718 and non-volatile storage drive 1720 can be configured to store the basic programming and data constructs that provide the functionality of various aspects of the present methods and devices, as described above.

Software instruction sets that provide the functionality of the present methods and devices can be stored in computer-readable medium 1712, RAM 1718, and/or non-volatile storage drive 1720. These instruction sets or code can be executed by the processor(s) 1714. Computer-readable medium 1712, RAM 1718, and/or non-volatile storage drive 1720 can also provide a repository to store data and data structures used in accordance with the present methods and devices. RAM 1718 and non-volatile storage drive 1720 can include a number of memories including a main random access memory (RAM) to store instructions and data during program execution and a read-only memory (ROM) in which fixed instructions are stored. RAM 1718 and non-volatile storage drive 1720 can include a file storage subsystem providing persistent (non-volatile) storage of program and/or data files. RAM 1718 and non-volatile storage drive 1720 can also include removable storage systems, such as removable flash memory.

Bus subsystem 1716 provides a mechanism to allow the various components and subsystems of computer 1702 communicate with each other as intended. Although bus subsystem 1716 is shown schematically as a single bus, alternative aspects of the bus subsystem can utilize multiple busses or communication paths within the computer 1702.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

### CONCLUSION

Whereas many alterations and modifications of the present methods and devices will no doubt become apparent to a person of ordinary skill in the art after having read the foregoing description, it is to be understood that the particular aspects shown and described by way of illustration are in no way intended to be considered limiting.

Moreover, the processes described above, as well as any other aspects of the disclosure, can each be implemented by software, but can also be implemented in hardware, firmware, or any combination of software, hardware, and firmware. Instructions for performing these processes can also be embodied as machine- or computer-readable code recorded on a machine- or computer-readable medium. In some aspects, the computer-readable medium can be a non-transitory computer-readable medium. Examples of such a non-transitory computer-readable medium include but are not limited to a read-only memory, a random-access memory, a flash memory, a CD-ROM, a DVD, a magnetic tape, a removable memory card, and optical data storage devices. In other aspects, the computer-readable medium can be a transitory computer-readable medium. In such aspects, the transitory computer-readable medium can be distributed over network-coupled computer systems so that the computer-readable code is stored and executed in a distributed fashion. For example, such a transitory computer-readable medium can be communicated from one electronic device to another electronic device using any suitable communications protocol. Such a transitory computer-readable medium can embody computer-readable code, instructions, data structures, program modules, or other data in a modulated data signal, such as a carrier wave or other transport mechanism, and can include any information delivery media. A modulated data signal can be a signal that has one or more of its characteristics set or changed in such a manner as to encode information in the signal.

It is to be understood that any or each module of any one or more of any system, device, or server can be provided as a software construct, firmware construct, one or more hardware components, or a combination thereof, and can be described in the general context of computer-executable instructions, such as program modules, that can be executed by one or more computers or other devices. Generally, a program module can include one or more routines, programs, objects, components, and/or data structures that can perform one or more particular tasks or that can implement one or more particular abstract data types. It is also to be understood that the number, configuration, functionality, and interconnection of the modules of any one or more of any system device, or server are merely illustrative, and that the number, configuration, functionality, and interconnection of existing modules can be modified or omitted, additional modules can be added, and the interconnection of certain modules can be altered.

While there have been described systems, methods, and computer-readable media for enabling efficient control of a media application at a media electronic device by a user electronic device, it is to be understood that many changes can be made therein without departing from the spirit and scope of the disclosure. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

It will be understood that terms such as “top,” “bottom,” “above,” “below,” and x-direction, y-direction, and z-direction as used herein as terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

The compositions and methods described herein are illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The aspects illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims.

Thus, it should be understood that although the present methods and compositions have been specifically disclosed by aspects and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

What is claimed is:

1. A cell separation system comprising multiple microfluidic devices with a balanced pressure across the multiple microfluidic devices, the system comprising:
an inlet for obtaining a stream of blood cells;
a plurality of microfluidic devices each configured to obtain a portion of the stream of blood cells from the inlet, wherein each of the plurality of microfluidic devices include:
at least one separation channel to separate cells in the stream of blood cells;
at least a first outlet for capturing released tumor cells included in the stream of blood cells; and
a second outlet for returning a remaining portion of the blood cells via a return line; and
a series of channels connecting the inlet to each of the plurality of microfluidic devices, wherein each of the channels include a length and a width that is variable to balance a pressure of each of the channels as the stream of blood cells are directed through each channel.

2. The cell separation system of claim 1, wherein the series of channels include at least:
a first channel connecting the inlet to a first microfluidic device, the first channel comprising a first length and a first width; and
a second channel connecting the inlet to a second microfluidic device, the second channel comprising a second length and a second width, wherein at least one of the second length and the second width is greater or less than that of the first channel.

3. The cell separation system of claim 1, wherein the length and width of each of the series of channels is selected based on a pressure loss due to friction loss as each portion of the stream of blood cells travel through each channel.

4. The cell separation system of claim 3, wherein determining the pressure loss is based on a dynamic viscosity, a length, a volumetric flow rate, and a cross sectional area of each of the series of channels.

5. The cell separation system of claim 1, wherein the length and width of each of the series of channels is based on a fluid density, a hydraulic diameter, a mean flow velocity, and a flow coefficient of each of the series of channels.

6. The cell separation system of claim 1, further comprising:
any one of a valve, a chamber, and a pump connected to each channel, wherein the valve, chamber, and pump are configured to modify and control the pressure of each channel.

7. The cell separation system of claim 6, further comprising:
a closed-loop control system configured to adjust the pressure of the pump based on detected pressure changes in any of the channels.

8. The cell separation system of claim 7, wherein a portion of the RTCs are configured to be held in a first chamber in a first channel of the series of channels that are pressurized by a first pump, wherein the closed-loop control system is configured to obtain a pressure level by a pressure sensor and instruct the first pump to modify the pressure level in the first chamber to maintain a specified pressure level.

9. The cell separation system of claim 6, further comprising:
a reservoir pump connected to a reservoir configured to capture the released tumor cells output from the first output, wherein the reservoir is configured to be pressurized.

10. The particle cell separation system of claim 1, wherein the inlet for obtaining a stream of blood cells is directly connected to a patient.

11. A device comprising:
an inlet for obtaining a stream of blood cells;
a plurality of microfluidic devices each configured to obtain a portion of the stream of blood cells from the inlet, wherein each of the plurality of microfluidic devices include:
at least one separation channel to separate cells in the stream of blood cells;
at least a first outlet for capturing released tumor cells included in the stream of blood cells; and
a second outlet for returning a remaining portion of the blood cells via a return line;
a series of channels connecting the inlet to each of the plurality of microfluidic devices, wherein each of the channels include a length and a width that is variable to balance a pressure of each of the channels as the stream of blood cells are directed through each channel; and
one or more of a valve, a chamber, and/a pump connected to each channel, wherein the one or more of valve, chamber, and/pump are configured to modify and control the pressure of each channel.

12. The device of claim 11, further comprising:
a closed-loop control system configured to adjust the pressure of the pump based on detected pressure changes in any of the channels

13. The device of claim 11, further comprising:
a pump connected to a reservoir configured to capture the released tumor cells output from the first output, wherein the reservoir is configured to be pressurized.

14. The device of claim 11, wherein the length and width of each of the series of channels is selected based on a pressure loss due to friction loss as each portion of the stream of blood cells travel through each channel.

15. The device of claim 11, wherein the length and width of each of the series of channels is selected based on a pressure loss due to friction loss as each portion of the stream of blood cells travel through each channel.

16. The device of claim 11, wherein the length and width of each of the series of channels is based on a fluid density, a hydraulic diameter, a mean flow velocity, and a flow coefficient of each of the series of channels.

17. The device of claim 11, wherein the inlet for obtaining a stream of blood cells is directly connected to a patient.

18. A method for separating released tumor cells from a stream of blood cells via multiple microfluidic devices, the method comprising:
obtaining, at an inlet, a stream of blood cells;
pressurizing a series of channels using one or more of a valve, chamber, and a pump;
directing portions of each of the stream of blood cells through each of the series of channels to corresponding microfluidic devices, wherein at least one separation channel for each microfluidic device is configured to separate cells in each portion of the stream of blood cells;
obtaining released tumor cells at a first outlet of each of the microfluidic devices; and
obtaining remaining portions of the stream of blood cells at a second outlet of each of the microfluidic devices.

19. The method of claim 18, further comprising:
pressurizing a reservoir using a reservoir pump; and
capturing the released tumor cells from the first outlet at the reservoir.

20. The method of claim 18, further comprising:
determining a pressure in each of a series of channels connecting an inlet to a plurality of microfluidic devices; and
modifying a length and a width for each of the series of channels based on the pressure in each of a series of channels.

21. The method of claim 20, wherein the length and width of each of the series of channels is selected based on a pressure loss due to friction loss as each portion of the stream of blood cells travel through each channel.

22. The method of claim 21, wherein determining the pressure loss is based on a dynamic viscosity, a length, a volumetric flow rate, and a cross sectional area of each of the series of channels.

23. The method of claim 18, wherein in the outlet is directly attached to a patient.
